(19) United States
(12) Patent Application Publication
(10) Pub. No.: US 2018/0346418 A1
(43) Pub. Date: Dec. 6, 2018

(54) KINASE ACTIVITY REGULATING COMPOUND INTERMEDIATES PREPARATION METHOD

(71) Applicants: CENTAURUS BIOPHARMA CO., LTD., Beijing (CN); CHIA TAI TIANQING PHARMACEUTICAL GROUP CO., LTD., Lianyungang City, Jiangsu Province (CN)

(72) Inventors: Jijun LI, Beijing (CN); Yan Zhu, Beijing (CN); Huting Wang, Beijing (CN); Yongxin Han, Beijing (CN); Ticong Huang, Lianyungang City (CN); Xinlu Li, Lianyungang City (CN); Rui Zhao, Lianyungang City (CN); Xiquan Zhang, Lianyungang City (CN)

(21) Appl. No.: 15/779,427

(22) PCT Filed: Nov. 25, 2016
(86) PCT No.: PCT/CN2016/107274

§ 371 Date: May 25, 2018

(30) Foreign Application Priority Data
Nov. 25, 2015 (CN) 201510835557.1

(51) Int. Cl. C07D 207/327; C07C 303/40; C07C 303/38; C07C 227/04

(52) U.S. Cl. CPC C07D 207/327; C07C 303/40; C07C 311/48; C07C 227/04; C07C 303/38

(57) ABSTRACT

The present application relates to a preparation method of intermediate compounds with formula II and formula III of a compound (N-{3-[3-(9H-purin-6-yl) pyridin-2-ylamino]-4-chloro-2-fluorophenyl}-3-fluoropropane-1-sulfonamide) for regulating kinase activity.

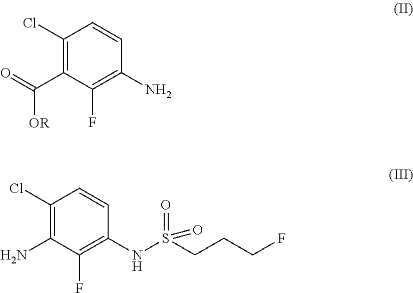

### CROSS-REFERENCE TO RELATED APPLICATIONS

This application claims the benefit of Chinese Invention Patent Application No. 201510835557.1 filed at the State Intellectual Property Office of the People's Republic of China on Nov. 26, 2015.

### TECHNICAL FIELD

The present application relates to methods for preparing intermediates of compounds for modulating kinase activity.

### BACKGROUND

N-{3-[3-(9H-purin-6-yl)pyridin-2-ylamino]-4-chloro-2-fluorophenyl}-3-fluoropropane-1-sulfonamide (Formula I) is a compound that modulates kinase activity, which can be used to treat diseases and disorders associated with the modulation of kinase activity. Example 9 in WO2013071865 disclosed the compound of Formula I and a preparation method thereof.

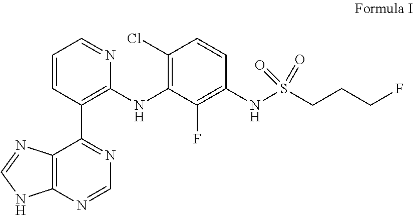 [SMILES: O=S(=O)(CCCF)Nc1ccc(Cl)c(Nc2ncccc2-c2ncnc3[nH]cnc23)c1F]

The preparation of the compound of Formula I involves a key intermediate represented by Formula II, 3-amino-6-chloro-2-fluorobenzoate compound, and the intermediate is used to prepare an intermediate represented by Formula III, N-(3-amino-4-chloro-2-fluorophenyl)-3-fluoropropane-1-sulfonamide, which is then reacted with 6-(2-fluoropyridin-3-yl)-9-(tetrahydro-2H-pyran-2-yl)-9H-purin, and remove a protecting group to obtain the compound of Formula I.

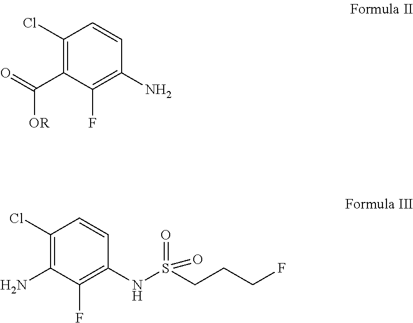

Scheme 4 in CN101808994A disclosed a method for preparing ethyl 3-amino-6-chloro-2-fluorobenzoate, in which a reaction temperature in several steps needs to be maintained below −70° C., and the steps are tedious, in which the reaction with ethyl chloroformate is performed for up to 64 hours, and the product needs to be purified by column chromatography. Therefore, this method is not suitable for industrial production.

CN102858754A disclosed a method for preparing the compound of Formula III. The yield of this method is only 46%, and the used reagent, DPPA, is expensive, and not suitable for industrial production, either.

Therefore, there is still a need for methods for preparing the compound of Formula II and the compound of Formula III to meet the requirements of industrial production.

### SUMMARY

In an aspect, the present application provides a method for preparing a compound of Formula II, comprising the following steps:

(1) reacting a compound of Formula IV with 2,5-hexanedione to prepare a compound of Formula V,

(2) reacting the compound of Formula V with a compound of Formula VII to prepare a compound of Formula VI, and

(3) reacting the compound of Formula VI to prepare the compound of Formula II,

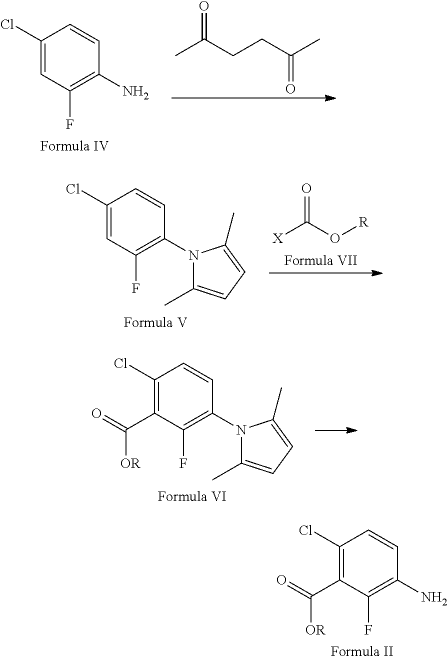

wherein R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted by one or more substituents selected from the group consisting of halogen, hydroxy, amino, lower alkyl, lower alkoxy and lower alkylthio;

preferably, R is selected from the group consisting of C₁-C₆ alkyl, C₃-C₆ cycloalkyl, C₃-C₆ heterocycloalkyl, aryl and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted by one or more substituents selected from the group consisting of halogen, hydroxy, amino, lower alkyl, lower alkoxy and lower alkylthio;

more preferably, R is selected from the group consisting of C₁-C₄ alkyl and benzyl, wherein each group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, halogen-substituted C₁-C₆ alkoxy, C₁-C₆ alkylthio, halogen-substituted C₁-C₆ alkylthio, and aryl and heteroaryl both of which are optionally substituted by one or more substituents selected from the group consisting of halogen, hydroxy, amino, C₁-C₆ alkyl, C₁-C₆ alkoxy and C₁-C₆ alkylthio; and

most preferably, R is selected from the group consisting of C₁-C₄ alkyl and benzyl, wherein each group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl, ethyl, and benzyl, wherein each group may be optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl, ethyl, and benzyl, wherein each group is optionally substituted with one or more substituents selected from the group consisting of fluorine, chlorine, bromine, methyl, ethyl, trifluoromethyl, and methoxyl.

In some specific embodiments of the present application, R is selected from ethyl or benzyl. In some more specific embodiments of the present application, the compound of Formula II is a compound of Formula II-0, and the compound of Formula VI is a compound of Formula VI-0.

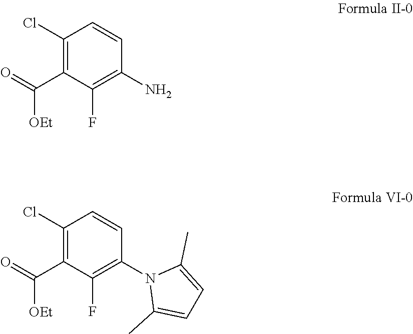 [SMILES: CCOC(=O)c1c(Cl)ccc(N)c1F ; CCOC(=O)c1c(Cl)ccc(-n2c(C)ccc2C)c1F]

In some specific embodiments of the present application, X in the compound of Formula VII is halogen, preferably chlorine and bromine, and most preferably chlorine.

It should be understood that X in the compound of Formula VII may also be other group(s), as long as the compound of Formula V is capable of reacting with the compound of Formula VII to prepare the compound of Formula VI.

Optionally, in the step (1) for preparing the compound of Formula V, a suitable catalyst may be selected as needed, and examples of the catalyst include, but are not limited to, p-toluenesulfonic acid, montmorillonite, β-cyclodextrine, 3,4-dihydroxy-3-cyclobutene-1,2-dione, sulfamic acid, trichloroacetic acid, a-amylase, and various metal catalysts, such as uranyl nitrate, bismuth nitrate, zirconium tetrachloride, zirconium dioxide, zirconium oxychloride, magnesium iodide, titanium dioxide, bismuth trichloride, lead(II) oxide, indium trifluoromethanesulfonate, ruthenium trichloride, ferriferous oxide, and so on. In some specific embodiments of the present application, the catalyst is preferably p-toluenesulfonic acid.

In some specific embodiments of the present application, in the step (1) for preparing the compound of Formula V, a suitable reaction solvent may be selected as needed, and the solvent is selected from one or more of the group consisting of water, methanol, ethanol, propanol, isopropanol, n-butanol, isobutanol, tert-butanol, 1,4-dioxane, formic acid, acetic acid, butanoic acid, pentanoic acid, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, preferably, one or more of the group consisting of toluene, tetrahydrofuran, dichloromethane, methanol, ethanol, acetonitrile, and water, and most preferably toluene. In some specific embodiments of the present application, in the step (1) for preparing the compound of Formula V, a suitable reaction temperature may be selected as needed. The reaction temperature is preferably from 0° C. to the boiling point of a reaction system, and more preferably, the boiling point of the reaction system.

In some specific embodiments of the present application, in the step (1) for preparing the compound of Formula V, a suitable molar ratio of the compound of Formula IV to 2,5-hexanedione may be selected as needed, and the molar ratio of the compound of Formula IV to 2,5-hexanedione may be 1:0.01 to 1:100, or 1:1 to 1:2. For example, in some specific embodiments of the present application, the molar ratio of the compound of Formula IV to 2,5-hexanedione is 1:0.9.

In some specific embodiments of the present application, in the step (2), the compound of Formula V can react with the compound of Formula VII in the presence of a base. A suitable base may be selected as needed, and the base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably n-butyllithium.

In some specific embodiments of the present application, in the step (2) for preparing the compound of Formula VI, a suitable reaction solvent may be selected as needed, and the solvent is selected from one or more of the group consisting of methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably tetrahydrofuran.

In some specific embodiments of the present application, in the step (2) for preparing the compound of Formula VI, a reaction temperature at the time of adding the base is not more than 25° C., preferably not more than 0° C., and most preferably not more than −30° C.

In some specific embodiments of the present application, in the step (2), the compound of Formula V may be first contacted with the base, and then the resulting mixture is contacted with the compound of Formula VII. In some specific embodiments of the present application, the step (2) may be carried out under the protection of nitrogen gas or argon gas.

In some specific embodiments of the present application, in the step (2) for preparing the compound of Formula VI, a suitable molar ratio of the compound of Formula V to the compound of Formula VII may be selected as needed. The molar ratio of the compound of Formula V to the compound of Formula VII may be 1:0.01 to 1:100, or 1:1 to 1:5. For example, in some specific embodiments of the present application, the molar ratio of the compound of Formula V to the compound of Formula VII is 1:2.

In some specific embodiments of the present application, in the step (3) for preparing the compound of Formula II, a suitable reaction solvent may be selected as needed. The solvent is selected from one or more of the group consisting of methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably a mixed solvent of ethanol and water.

In some specific embodiments of the present application, in the step (3) for preparing the compound of Formula II, a suitable reagent may be added as needed. The reagent includes, but is not limited to, hydroxylamine hydrochloride, hydroxylamine hydrochloride and a base, and hydrochloric acid and a base, wherein the base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyl lithium, lithium tert-butoxide, lithium diisopropylamide, sodium carbonate, potassium carbonate, sodium acetate, potassium acetate, sodium bicarbonate, and potassium bicarbonate. When the reagent is hydrochloric acid and a base, hydrochloric acid is first added to react for a period of time, and then the base is added. The period of time is not particularly limited, and a suitable period of time may be selected as needed. In some specific embodiments of the present application, the reagent is preferably hydroxylamine hydrochloride and triethylamine.

In some specific embodiments of the present application, in the step (3) for preparing the compound of Formula II, a reaction temperature is from 0° C. to the boiling point of a reaction system, and preferably 80° C.

In another aspect, the present application provides a method for preparing a compound of Formula VIII, comprising reacting a compound of Formula II with 3-fluoro-1-propylsulfonyl chloride to prepare the compound of Formula VIII,

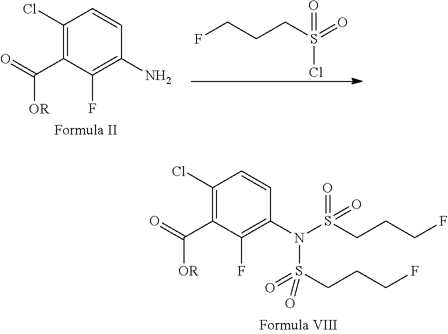

wherein R is defined as above.

In some specific embodiments of the present application, 3-fluoro-1-propylsulfonyl chloride may be prepared by a conventional method in the prior art, for example, prepared by the following method:

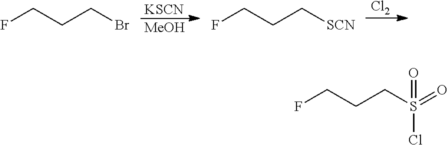

In some specific embodiments of the present application, the compound of Formula VIII is preferably a compound of Formula VIII-0,

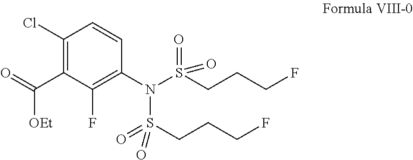 [SMILES: CCOC(=O)c1c(Cl)ccc(N(S(=O)(=O)CCCF)S(=O)(=O)CCCF)c1F]

In some specific embodiments of the present application, in the reaction for preparing the compound of Formula VIII, a suitable molar ratio of the compound of Formula II to 3-fluoro-1-propylsulfonyl chloride may be selected as needed. For example, the molar ratio of the compound of Formula II to 3-fluoro-1-propylsulfonyl chloride may be 1:2 to 1:5. The molar ratio of the compound of Formula II to 3-fluoro-1-propylsulfonyl chloride is preferably 1:2.

In some specific embodiments of the present application, in the reaction for preparing the compound of Formula VIII, a suitable base may be selected as needed. The base may be selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably triethylamine or pyridine.

In some specific embodiments of the present application, in the reaction for preparing the compound of Formula VIII, a suitable solvent may be selected as needed. The solvent is selected from one or more of the group consisting of methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably dichloromethane.

In some specific embodiments of the present application, when 3-fluoro-1-propylsulfonyl chloride is added, a reaction temperature is not more than 40° C., preferably not more than 30° C., and most preferably not more than 20° C.

Optionally, the reaction for preparing the compound of Formula VIII from the compound of Formula II may be carried out under the protection of nitrogen gas or argon gas.

In another aspect, the present application provides a method for preparing a compound of Formula VIIIa, comprising reacting a compound of Formula II with 3-fluoro-1-propylsulfonyl chloride to prepare the compound of Formula VIIIa,

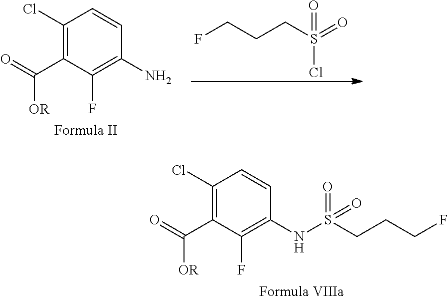

wherein R is defined as above.

In some specific embodiments of the present application, the compound of Formula VIIIa is preferably a compound of Formula VIIIa-0,

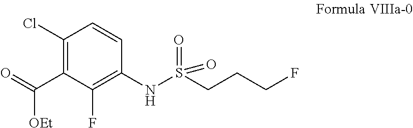 [SMILES: CCOC(=O)c1c(Cl)ccc(NS(=O)(=O)CCCF)c1F]

In some specific embodiments of the present application, in the reaction for preparing the compound of Formula VIIIa, a suitable molar ratio of the compound of Formula II to 3-fluoro-1-propylsulfonyl chloride may be selected as needed. For example, the molar ratio of the compound of Formula II to 3-fluoro-1-propylsulfonyl chloride may be 1:1 to 1:1.2. The molar ratio of the compound of Formula II to 3-fluoro-1-propyl sulfonyl chloride is preferably 1:1.2.

In some specific embodiments of the present application, in the reaction for preparing the compound of Formula VIIIa, a suitable base may be selected as needed. The base may be selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably triethylamine or pyridine.

In some specific embodiments of the present application, in the reaction for preparing the compound of Formula VIIIa, a suitable solvent may be selected as needed. The solvent is selected from one or more of the group consisting of methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably dichloromethane.

In some specific embodiments of the present application, in the reaction for preparing the compound of Formula VIIIa, when 3-fluoro-1-propylsulfonyl chloride is added, a reaction temperature is not more than 40° C., preferably not more than 30° C., and most preferably not more than 25° C.

Optionally, the reaction for preparing the compound of Formula VIIIa from the compound of Formula II may be carried out under the protection of nitrogen gas or argon gas.

In another aspect, the present application provides a method for preparing a compound of Formula IX, comprising reacting a compound of Formula VIII to prepare the compound of Formula IX,

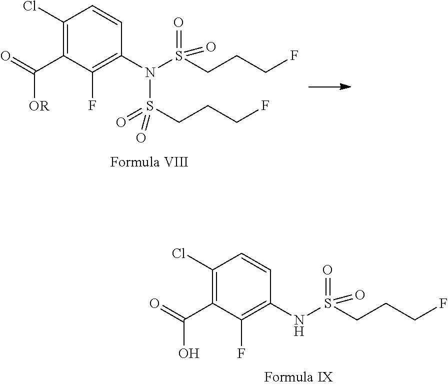

In some specific embodiments of the present application, in the reaction for preparing the compound of Formula IX from the compound of Formula VIII, a suitable base may be first added as needed, followed by the addition of a suitable acid. The base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably sodium hydroxide or potassium hydroxide. The acid is selected from various organic and inorganic acids, such as hydrochloric acid, sulfuric acid, nitric acid, phosphoric acid, maleic acid, citric acid, and so on.

In some specific embodiments of the present application, in the reaction for preparing the compound of Formula IX from the compound of Formula VIII, a reaction temperature at the time of adding the base is below 20° C., preferably below 0° C., and most preferably below 10° C.

In some specific embodiments of the present application, in the reaction for preparing the compound of Formula IX from the compound of Formula VIII, a reaction temperature at the time of adding the acid is below 20° C., preferably below 0° C., and most preferably below 10° C.

In some specific embodiments of the present application, in the reaction for preparing the compound of Formula IX from the compound of Formula VIII, the acid is added to make a pH value of a reaction system not more than 7, preferably not more than 5, and most preferably not more than 3.

In some specific embodiments of the present application, in the reaction for preparing the compound of Formula IX from the compound of Formula VIII, a suitable solvent may be selected as needed. The solvent is selected from one or more of the group consisting of water, methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably tetrahydrofuran.

In another aspect, the present application provides a method for preparing a compound of Formula IX, comprising reacting a compound of Formula VIIIa to prepare the compound of Formula IX,

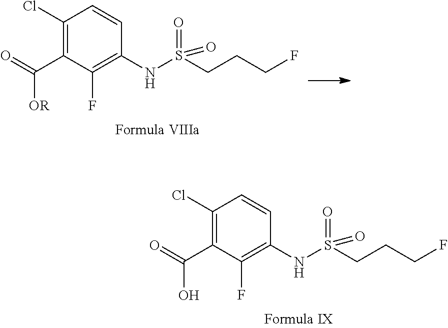

In some specific embodiments of the present application, in the reaction for preparing the compound of Formula IX from the compound of Formula VIIIa, a suitable base may be added as needed, followed by the addition of a suitable acid. The base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, pyridine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably sodium hydroxide or potassium hydroxide. The acid is selected from various organic and inorganic acids, such as hydrochloric acid, sulfuric acid, nitric acid, phosphoric acid, maleic acid, citric acid and so on.

In some specific embodiments of the present application, in the reaction for preparing the compound of Formula IX from the compound of Formula VIIIa, a reaction temperature at the time of adding the base is below 20° C., preferably below 0° C., and most preferably below 10° C.

In some specific embodiments of the present application, in the reaction for preparing the compound of Formula IX from the compound of Formula VIIIa, a reaction temperature at the time of adding the acid is below 20° C., preferably below 0° C., and most preferably below 10° C.

In some specific embodiments of the present application, in the reaction for preparing the compound of Formula IX from the compound of Formula VIIIa, the acid is added to make a pH value of a reaction system not more than 7, preferably not more than 5, and most preferably not more than 3.

In some specific embodiments of the present application, in the reaction for preparing the compound of Formula IX from the compound of Formula VIIIa, a suitable solvent may be selected as needed. The solvent is selected from one or more of the group consisting of water, methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably tetrahydrofuran.

In another aspect, the present application provides a method for preparing a compound of Formula X, comprising reacting a compound of Formula IX with N,N′-carbonyldiimidazole (CDI), and then adding NH₃ to prepare the compound of Formula X,

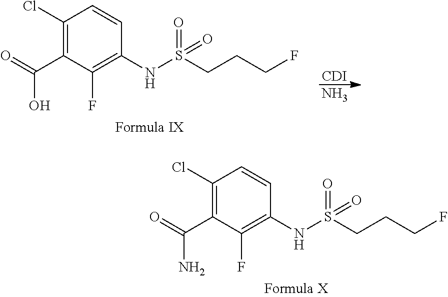

In some specific embodiments of the present application, in the reaction for preparing the compound of Formula X, a suitable solvent may be selected as needed. The solvent is selected from one or more of the group consisting of water, methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, and preferably tetrahydrofuran.

In some specific embodiments of the present application, in the reaction for preparing the compound of Formula X, NH₃ may be added in the form of ammonia gas or ammonia water.

In some specific embodiments of the present application, in the reaction for preparing the compound of Formula X, the compound of Formula IX is reacted with N,N′-carbonyldiimidazole (CDI) at a temperature of from 0° C. to the boiling point of a reaction system, preferably the boiling point of the reaction system.

In some specific embodiments of the present application, in the reaction for preparing the compound of Formula X, NH₃ is added at a temperature of not more than 25° C., preferably not more than 5° C., and most preferably not more than 0° C. In some specific embodiments of the present application, NH₃ is added at a temperature of 0° C.

In some specific embodiments of the present application, the method for preparing the compound of Formula X further comprises a step of adding an acid following the step of adding NH₃ and reacting for a period of time. The acid is selected from various organic and inorganic acids, such as hydrochloric acid, sulfuric acid, nitric acid, phosphoric acid, maleic acid, citric acid, and so on, and preferably hydrochloric acid. In some specific embodiments of the present application, the acid is added to make a pH value of a reaction system not more than 7, preferably not more than 5, and most preferably not more than 3.

In yet another aspect, the present application provides a method for preparing a compound of Formula III, comprising reacting a compound of Formula X to prepare the compound of Formula III,

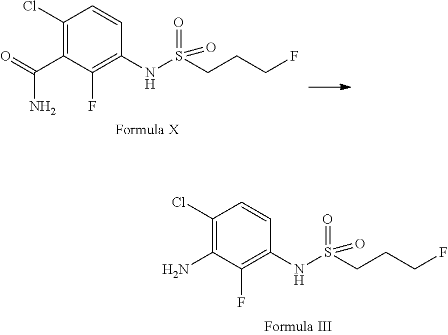 [SMILES: NC(=O)c1c(Cl)ccc(NS(=O)(=O)CCCF)c1F ; Nc1c(Cl)ccc(NS(=O)(=O)CCCF)c1F]

In some specific embodiments of the present application, the method for preparing the compound of Formula III preferably comprises the following steps:

i. reacting the compound of Formula X in the presence of a base and hypochlorite;

ii. adding thiosulfate to the reaction system of step i; and

iii. adding an acid to the reaction system of step ii to obtain the compound of Formula III.

In some specific embodiments of the present application, in the method for preparing the compound of Formula III, a suitable solvent may be selected as needed. The solvent is selected from one or more of the group consisting of water, methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO, preferably one or both of tetrahydrofuran and water, and most preferably a mixed solvent of tetrahydrofuran and water.

In some specific embodiments of the present application, in the step i of the method for preparing the compound of Formula III, a suitable base may be selected as needed. The base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably sodium hydroxide or potassium hydroxide.

In some specific embodiments of the present application, in and step i of the method for preparing the compound of Formula III, the compound of Formula X is first contacted with the base, and then with hypochlorite.

In some specific embodiments of the present application, a reaction temperature when contacting with hypochlorite is not more than 25° C., preferably not more than 5° C.

In some specific embodiments of the present application, the reaction temperature is raised after contacting with hypochlorite. For example, the reaction temperature is raised to 25° C.

In some specific embodiments of the present application, in the step ii of the method for preparing the compound of Formula III, thiosulfate is added at a temperature of 25° C.

In some specific embodiments of the present application, hypochlorite is preferably sodium hypochlorite or potassium hypochlorite, and thiosulfate is preferably sodium thiosulfate or potassium thiosulfate.

In some specific embodiments of the present application, in the step iii of the method for preparing the compound of Formula III, the acid is selected from various organic and inorganic acids, such as hydrochloric acid, sulfuric acid, nitric acid, phosphoric acid, maleic acid, citric acid, and so on, and preferably hydrochloric acid.

In some specific embodiments of the present application, in the step iii of the method for preparing the compound of Formula III, the acid is added to make a pH value of a reaction system not more than 7, preferably not more than 6, and more preferably between 5 to 6.

In some specific embodiments of the present application, in the step iii of the method for preparing the compound of Formula III, the reaction system is maintained at a temperature of not more than 40° C., preferably not more than 30° C., and most preferably not more than 20° C.

In some specific embodiments of the present application, the method for preparing the compound of Formula III further comprises a step of adding ethanol or isopropanol following the step iii.

In yet another aspect, the present application provides a method for preparing a compound of Formula III, comprising the following steps:

(a) reacting a compound of Formula IX with N,N′-carbonyldiimidazole (CDI), and then adding NH₃ to prepare a compound of Formula X, and

(b) reacting the compound of Formula X to prepare the compound of Formula III,

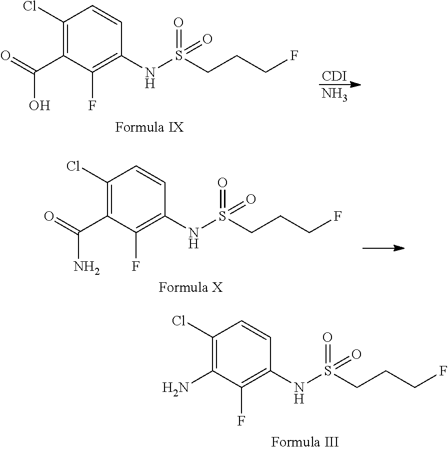

In some specific embodiments of the present application, in the method for preparing the compound of Formula III, a suitable solvent may be selected as needed. The solvent is selected from one or more of the group consisting of water, methanol, ethanol, propanol, isopropanol, n-butanol, isobutanol, tert-butanol, 1,4-dioxane, acetone, butanone, pentanone, cyclopentanone, hexanone, cyclohexanone, diethyl ether, ethyl acetate, butyl acetate, tetrahydrofuran, acetonitrile, benzene, toluene, xylene, DMF, DMAC, and DMSO.

In the step (a), the solvent is preferably tetrahydrofuran. In the step (b), the solvent is preferably one or both of tetrahydrofuran and water, and most preferably a mixed solvent of tetrahydrofuran and water.

In some specific embodiments of the present application, in the step (a) of the method for preparing the compound of Formula III, NH₃ may be added in the form of ammonia gas or ammonia water.

In some specific embodiments of the present application, in the step (a) of the method for preparing the compound of Formula III, the compound of Formula IX is reacted with N,N′-carbonyldiimidazole (CDI) at a temperature of from 0° C. to the boiling point of a reaction system, preferably the boiling point of the reaction system.

In some specific embodiments of the present application, in the step (a) of the method for preparing the compound of Formula III, NH₃ is added at a reaction temperature of not more than 25° C., preferably not more than 5° C., more preferably not more than 0° C., and most preferably 0° C.

In some specific embodiments of the present application, the step (a) of the method for preparing a compound of Formula III further comprises a step of adding an acid, and the acid is selected from various organic and inorganic acids, such as hydrochloric acid, sulfuric acid, nitric acid, phosphoric acid, maleic acid, citric acid, and so on, and preferably hydrochloric acid.

In some specific embodiments of the present application, in the method for preparing the compound of Formula III, the acid is added to make a pH value of a reaction system not more than 7, preferably not more than 5, and most preferably not more than 3.

In some specific embodiments of the present application, the step (b) of the method for preparing the compound of Formula III comprises the following steps:

i. reacting the compound of Formula X in the presence of a base and hypochlorite;

ii. adding thiosulfate to the reaction system of step i; and

iii. adding an acid to the reaction system of step ii.

In some specific embodiments of the present application, in the step i, a suitable base may be selected as needed. The base is selected from one or more of the group consisting of sodium hydroxide, potassium hydroxide, sodium methoxide, sodium ethoxide, sodium n-propoxide, sodium isopropoxide, sodium n-butoxide, sodium tert-butoxide, N,N-diisopropylethylamine, triethylamine, diethylamine, ethylenediamine, cesium carbonate, lithium carbonate, sodium hydride, sodium amide, n-butyllithium, lithium tert-butoxide, lithium diisopropylamide, sodium carbonate, potassium carbonate, sodium acetate, potassium acetate, sodium bicarbonate, and potassium bicarbonate, and preferably sodium hydroxide or potassium hydroxide.

In some specific embodiments of the present application, in the step i, the compound of Formula X is first contacted with the base, and then with hypochlorite.

In some specific embodiments of the present application, in the step i, a reaction temperature when contacting with hypochlorite is not more than 25° C., and preferably not more than 5° C.

In some specific embodiments of the present application, in the step i, the reaction temperature is raised after contacting with hypochlorite. For example, the reaction temperature is raised to 25° C.

In some specific embodiments of the present application, in the step ii, thiosulfate is added at a reaction temperature of 25° C.

In some specific embodiments of the present application, hypochlorite is sodium hypochlorite or potassium hypochlorite.

In some specific embodiments of the present application, thiosulfate is sodium thiosulfate or potassium thiosulfate.

In some specific embodiments of the present application, in the step iii, the acid is selected from various organic and inorganic acids, such as hydrochloric acid, sulfuric acid, nitric acid, phosphoric acid, maleic acid, citric acid, and so on, and preferably hydrochloric acid.

In some specific embodiments of the present application, in the step iii, the acid is added to make a pH value of a reaction system not more than 7, preferably not more than 6, and more preferably between 5 to 6.

In some specific embodiments of the present application, in the step iii, the reaction system is maintained at a temperature of not more than 40° C., preferably not more than 30° C., and most preferably not more than 20° C.

In some specific embodiments of the present application, the preparing of the compound of Formula III further comprises a step of adding ethanol or isopropanol following the step iii.

In yet another aspect, the present application provides a method for preparing a compound of Formula III, comprising the following steps:

(1) reacting a compound of Formula IV with 2,5-hexanedione to prepare a compound of Formula V,

(2) reacting the compound of Formula V with a compound of Formula VII to prepare a compound of Formula VI,

(3) reacting the compound of Formula VI to prepare a compound of Formula II,

(4) reacting the compound of Formula II with 3-fluoro-1-propylsulfonyl chloride to prepare a compound of Formula VIII,

(5) reacting the compound of Formula VIII to prepare a compound of Formula

(6) reacting the compound of Formula IX with N,N′-carbonyldiimidazole (CDI), and then adding NH₃ to prepare a compound of Formula X, and

(7) reacting the compound of Formula X to prepare the compound of Formula III.

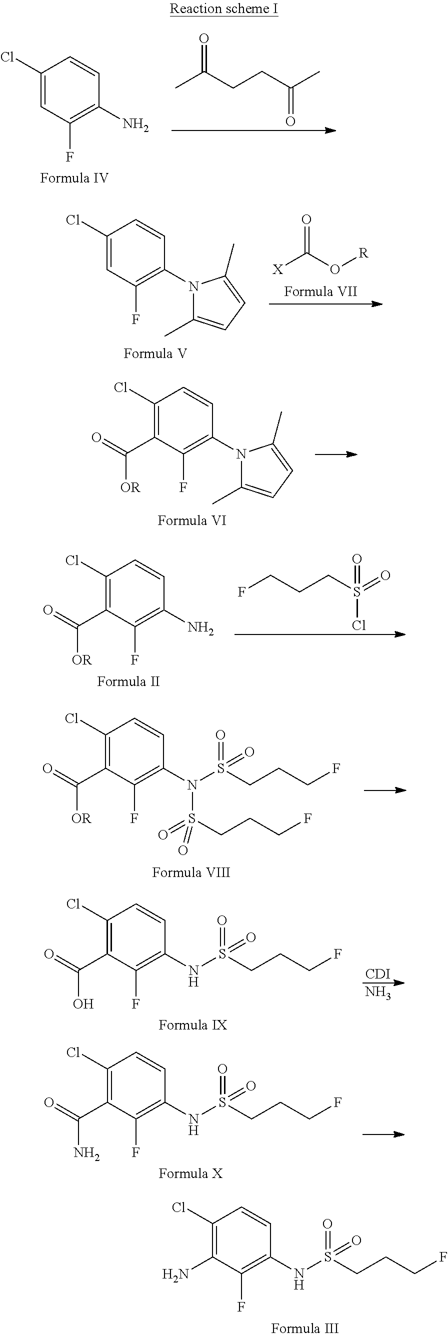

In yet another aspect, the present application provides a method for preparing a compound of Formula III, comprising the following steps:

(1) reacting a compound of Formula IV with 2,5-hexanedione to prepare a compound of Formula V,

(2) reacting the compound of Formula V with a compound of Formula VII to prepare a compound of Formula VI,

(3) reacting the compound of Formula VI to prepare a compound of Formula II,

(4) reacting the compound of Formula II with 3-fluoro-1-propylsulfonyl chloride to prepare a compound of Formula VIIIa,

(5) reacting the compound of Formula VIIIa to prepare a compound of Formula IX,

(6) reacting the compound of Formula IX with N,N′-carbonyldiimidazole (CDI), and then adding NH₃ to prepare a compound of Formula X, and

(7) reacting the compound of Formula X to prepare the compound of Formula III.

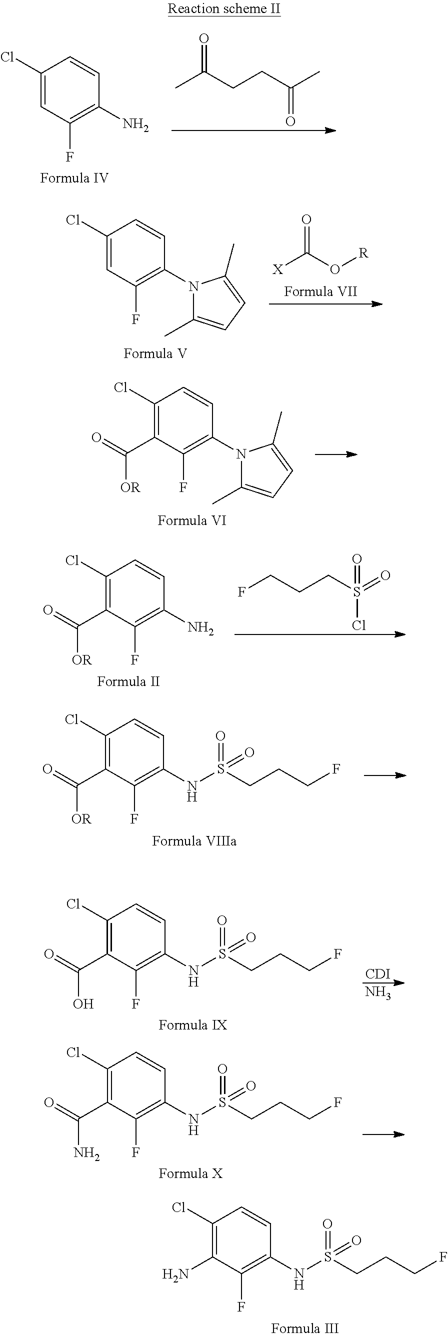

In another aspect, the present application provides a compound of Formula VI, or a salt or solvate thereof,

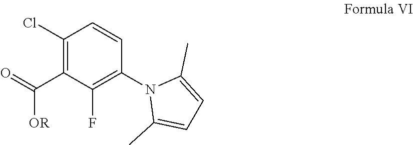

wherein, R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted with one or more substituents selected from the group consisting of halogen, hydroxy, amino, lower alkyl, lower alkoxy and lower alkylthio;

preferably, R is selected from the group consisting of C₁-C₆ alkyl, C₃-C₆ cycloalkyl, C₃-C₆ heterocycloalkyl, aryl and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted with one or more substituents selected from the group consisting of halogen, hydroxy, amino, lower alkyl, lower alkoxy and lower alkylthio;

more preferably, R is selected from the group consisting of C₁-C₄ alkyl and benzyl, wherein each group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, halogen-substituted C₁-C₆ alkoxy, C₁-C₆ alkylthio, halogen-substituted C₁-C₆ alkylthio, and aryl and heteroaryl both of which are optionally substituted by one or more substituents selected from the group consisting of halogen, hydroxy, amino, C₁-C₆ alkyl, C₁-C₆ alkoxy and C₁-C₆ alkylthio; and

most preferably, R is selected from the group consisting of C₁-C₄ alkyl and benzyl, wherein each group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl, ethyl, and benzyl, wherein each group may be optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl, ethyl, and benzyl, wherein each group is optionally substituted with one or more substituents selected from the group consisting of fluorine, chlorine, bromine, methyl, ethyl, trifluoromethyl and methoxyl.

In some specific embodiments of the present application, R is selected from the group consisting of ethyl and benzyl.

In still yet another aspect, the present application provides a compound of Formula VIII or VIIIa, or a salt or solvate thereof,

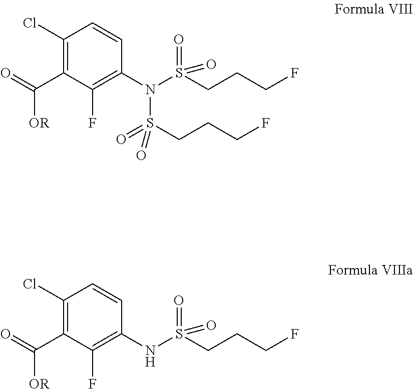

wherein, R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and heteroaryl which is optionally substituted with one or more substituents selected from the group consisting of halogen, hydroxy, amino, lower alkyl, lower alkoxy, and lower alkylthio.

Preferably, R is selected from the group consisting of C₁-C₆ alkyl, C₃-C₆ cycloalkyl, C₃-C₆ heterocycloalkyl, aryl and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and heteroaryl which is optionally substituted with one or more substituents selected from the group consisting of halogen, hydroxy, amino, lower alkyl, lower alkoxy, and lower alkylthio.

More preferably, R is C₁-C₄ alkyl, wherein the group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, halogen-substituted C₁-C₆ alkoxy, C₁-C₆ alkylthio, halogen-substituted C₁-C₆ alkylthio, and heteroaryl which is optionally substituted with one or more substituents selected from the group consisting of halogen, hydroxy, amino, C₁-C₆ alkyl, C₁-C₆ alkoxy, and C₁-C₆ alkylthio.

Most preferably, R is selected from the group consisting of C₁-C₄ alkyl and benzyl, wherein each group is optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl and ethyl, wherein each group may be optionally substituted with one or more substituents selected from the group consisting of halogen, C₁-C₆ alkyl, halogen-substituted C₁-C₆ alkyl, C₃-C₆ cycloalkyl, halogen-substituted C₃-C₆ cycloalkyl, C₁-C₆ alkoxy, and halogen-substituted C₁-C₆ alkoxy.

In some specific embodiments of the present application, R is selected from the group consisting of methyl and ethyl, wherein each group is optionally substituted with one or more substituents selected from the group consisting of fluorine, chlorine, bromine, methyl, ethyl, trifluoromethyl, and methoxyl.

In some specific embodiments of the present application, R is ethyl.

In still yet another aspect, the present application provides a compound of Formula X, or a salt or solvate thereof,

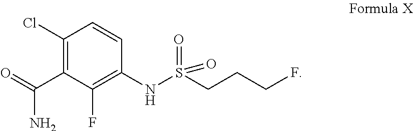 [SMILES: NC(=O)c1c(Cl)ccc(NS(=O)(=O)CCCF)c1F]

In still yet another aspect, the present application provides use of a compound of Formula VI, a compound of Formula VIII, a compound of Formula VIIIa, or a compound of Formula X, or a salt or solvate thereof in the preparation of N-(3-amino-4-chloro-2-fluorophenyl)-3-fluoropropane-1-sulfonamide.

In still yet another aspect, the present application provides use of a compound of Formula VI, a compound of Formula VIII, a compound of Formula VIIIa, or a compound of Formula X, or a salt or solvate thereof in the preparation of N-{3-[3-(9H-purin-6-yl)pyridin-2-ylamino]-4-chloro-2-fluorophenyl}-3-fluoropropane-1-sulfonamide.

In the preparation methods of the present application, 3-amino-6-chloro-2-fluorobenzoate compounds represented by Formula II are obtained by preparing 1-(4-chloro-2-fluorophenyl)-2,5-dimethyl-1H-pyrrole from 4-chloro-2-fluoroaniline, then reacting with the corresponding acyl halide to link an ester group, and then performing a further reaction. The preparation methods have the following advantages: easy access to raw materials and reagents, mild reaction conditions, short reaction period, friendly to the environment, simple preparation steps and ease to operate, and high yield. N,N′-carbonyldiimidazole and NH₃ are used in the preparation of N-(3-amino-4-chloro-2-fluorophenyl)-3-fluoropropane-1-sulfonamide, which are cheap and easy to obtain, and the preparation steps are simple and easy to operate. Not only the yield is obviously increased, but also the product has a high purity. Therefore, the preparation methods described in the present application are particularly suitable for industrial production.

### TERMS AND DEFINITIONS

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change, and particular information on the internet can come and go, but equivalent information can be found by searching the internet or other appropriate reference source. Reference thereto evidences the availability and public dissemination of such information.

It should be understood that the foregoing general description and the following detailed description are exemplary and explanatory only, and are not restrictive of any subject matter claimed. In this application, the use of the singular includes the plural, unless specifically stated otherwise. It must be noted that, as used in the specification and the appended claims, the singular forms “a”, “an” and “the” include plural referents, unless the context clearly dictates otherwise. It should also be noted that use of “or” means “and/or” unless stated otherwise. Furthermore, use of the term “including” as well as other forms, such as “include”, “includes”, and “included” is not limiting.

Unless otherwise noted, the use of general chemical terms, such as but not limited to “alkyl” and “aryl”, is equivalent to their optionally substituted forms. For example, “alkyl” as used herein, includes optionally substituted alkyl.

The term “optional” or “optionally” means that the subsequently described event or circumstance may or may not occur, and that the description includes instances where said event or circumstance occurs and instances where said event or circumstance does not occurs. For example, “optionally substituted alkyl” as defined below means either “alkyl” or “substituted alkyl”. Further, an optionally substituted group may be un-substituted (e.g., CH₂CH₃), fully substituted (e.g., CF₂CF₃), mono-substituted (e.g., CH₂CH₂F) or substituted at a level anywhere in-between fully substituted and mono-substituted (e.g., CH₂CHF₂, CF₂CH₃, CFHCHF₂, etc).

As used herein, includes C₁-C₂, C₁-C₃ . . . C₁-Cₙ. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Therefore, by way of example only, “C₁-C₄ alkyl” indicates that there are one to four carbon atoms in the alkyl group, i.e., the alkyl group is selected from the group consisting of methyl, ethyl, propyl, isopropyl, n-butyl, isobutyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “hetero” as used herein, alone or in combination, refer to an atom other than carbon and hydrogen. Heteroatoms are independently selected from the group consisting of oxygen, nitrogen, sulfur, phosphorous, silicon, selenium and tin, but are not limited to these atoms. Where there are two or more heteroatoms in embodiments, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain univalent saturated hydrocarbyl having, for example, from one to about eighteen, or one to about ten carbon atoms, and more preferably one to six carbon atoms. The term “lower alkyl” as used herein, alone or in combination, refers to an alkyl having relatively less carbon atoms, for example having one to about eight carbon atoms, preferably having one to about six, or one to about four carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The “alkyl” as used in combination includes, but not limited to, the “alkyl” included in “alkoxy”, “alkylthio”, “mono-alkylamino” and “di-alkylamino”, etc.

The term “alkenyl” as used herein, alone or in combination, refers to an optionally substituted straight-chain, or optionally substituted branched-chain univalent hydrocarbyl having one or more carbon-carbon double-bonds and having, for example, from two to about eighteen or two to about ten carbon atoms, and more preferably two to about six carbon atoms. The group may be in either the cis or trans conformation about the double bond(s), and should be understood to include both isomers. The term “lower alkenyl” as used herein, alone or in combination, refers to an alkenyl having relatively less carbon atoms, for example having two to about eight carbon atoms, preferably having two to about six, or two to about four carbon atoms. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkynyl” as used herein, alone or in combination, refers to an optionally substituted straight-chain or optionally substituted branched-chain univalent hydrocarbyl having one or more carbon-carbon triple-bonds and having, for example, from two to about eighteen or two to about ten carbon atoms, and more preferably from two to about six carbon atoms. The term “lower alkynyl” as used herein, alone or in combination, refers to an alkynyl having relatively less carbon atoms, for example having two to about eight carbon atoms, preferably having two to about six, or two to about four carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C2-6 alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “membered” is meant to denote the number of skeletal atoms that constitute the ring. Therefore, by way of example only, cyclohexane, pyridine, pyrane and pyrimidine are six-membered rings and cyclopentane, pyrrole, tetrahydrofuran and thiophene are five-membered rings.

The term “cycloalkyl” as used herein, alone or in combination, refers to an optionally substituted, saturated, univalent hydrocarbon ring, containing from three to about fifteen ring carbon atoms or from three to about ten ring carbon atoms, though may include additional, non-ring carbon atoms as substituents (e.g. methyl cyclopropyl). The cycloalkyl may have three to about ten, or three to about eight, or three to about six, or three to five ring atoms. The examples include but not limited to cyclopropyl, cyclobutyl, cyclopentyl, and cyclohexyl.

The term “lower cycloalkyl” as used herein, alone or in combination, refers to those having relatively less ring atoms, for example, having five to about ten or five to about eight, or five to six ring atoms, or three to six ring atoms, for example, having three, four, five or six ring atoms.

The term “heterocycloalkyl” refers to a fully-saturated cyclic group in the form of monocycle, bicycle, or spirocycle. Unless indicated otherwise, the heterocycle is typically a 3 to 7-membered ring containing 1 to 3 heteroatoms (preferably 1 or 2 heteroatoms) independently selected from sulfur, oxygen and/or nitrogen. Non-limiting examples of “heterocycloalkyl” include azetidinyl, oxetanyl, thietanyl, homopiperidinyl, oxepanyl, thiepanyl, dioxanyl, 1,3-dioxolanyl, dithianyl, dithiolanyl, pyrazolidinyl, imidazolidinyl, 3-azabicyclo[3.1.0]hexyl and 3-azabicyclo[4.1.0]heptyl, and so on.

The term “aryl” as used herein, alone or in combination, refers to a full carbon monocyclic or fused ring having a fully conjugated pi-electron system and having from 6 to 14 carbon atoms, preferably from 6 to 12 carbon atoms, and most preferably having 6 carbon atoms. Non-limiting examples of unsubstituted aryl include, but are not limited to, phenyl, naphthyl and anthryl.

The term “heteroaryl” as used herein, alone or in combination, refers a monocyclic or fused ring having 5 to 12 ring atoms, such as, 5, 6, 7, 8, 9, 10, 11 or 12 ring atoms, wherein 1, 2, 3 or 4 ring atoms are selected from the group consisting of N, O and S, and the rest of ring atom(s) is(are) carbon atom(s), and the ring has a fully conjugated pi-electron system. The heteroaryl may be unsubstituted or substituted, and the substituent includes, but is not limited to, alkyl, alkoxy, aryl, aralkyl, amino, halogen, hydroxy, cyano, nitro, carbonyl, heteroalicyclic group. Non-limiting examples of unsubstituted heteroaryl include, but are not limited to, pyrrolyl, furyl, thienyl, imidazolyl, oxazolyl, pyrazolyl, pyridyl, pyrimidyl, pyrazinyl, quinolyl, isoquinolyl, tetrazolyl, and triazinyl.

The terms “halogen”, “halo” or “halide” as used herein, alone or in combination refer to fluoro, chloro, bromo and iodo.

The term “alkoxy” as used herein, alone or in combination, refers to “-0-alkyl.” Non-limiting examples of alkoxy include methoxy, ethoxy, n-propoxy, isopropoxy, n-butoxy, isobutoxy, sec-butoxy and tert-butoxy, and the like.

The term “alkylthio” as used herein, alone or in combination, refers to “-S-alkyl.” Non-limiting examples of alkylthio include methylthio, ethylthio, propylthio, butylthio, and the like.

The term “lower alkyl”, “lower alkoxy” and “lower alkylthio” as used herein, alone or in combination, refers to those having one to about eight, or one to six, or one to five, or one to four, or one to three or one to two carbon atoms.

Examples of the term “salt” as used herein include salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyne-1,4-dioate, camphorate, camphorsulfonate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate, undeconate and xylenesulfonate.

The term “solvate” as used herein refers to a combination of a compound of this invention with a solvent molecule formed by solvation. In some situations, the solvate refers to a hydrate, i.e., the solvent molecule is a water molecule, the combination of a compound of this invention and water forms a hydrate.

### EXAMPLES

### Example 1

### Preparation of 1-(4-chloro-2-fluorophenyl)-2,5-dimethyl-1H-pyrrole (Formula V)

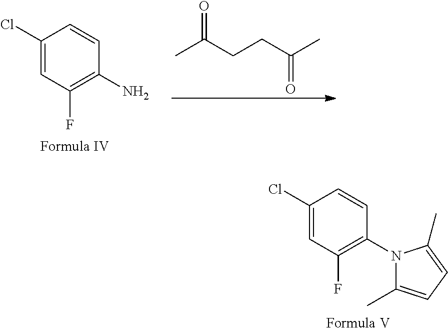

To a 5 L three-necked reaction flask equipped with a water segregator was added 4-chloro-2-fluoroaniline (598 g, 4.11 mol), 2,5-hexanedione (518 g, 4.54 mol) and toluene (3.0 L), and stirred for 10 minutes until the system was uniformly mixed. A catalytic amount of p-toluenesulfonic acid (1.4 g) was added, and heated under reflux for 2 hours. After cooling to room temperature, the system was washed successively with water (1 L) and saturated brine (1 L), and was dried over anhydrous sodium sulfate. After the desiccant was filtered off, the solvent was removed through concentration in vacuo. The resulting residue was concentrated under reduced pressure to obtain a colourless and clear liquid (850 g, 92% yield). The liquid product was rapidly solidified upon cooling.

¹H NMR (CDCl₃):δ7.28-7.18 (3H, m), 5.93 (2H, s), 2.00 (6H, s).

### Example 2

### Preparation of ethyl 6-chloro-3-(2,5-dimethyl-1H-pyrrol-1-yl)-2-fluorobenzoate (Formula VI-0)

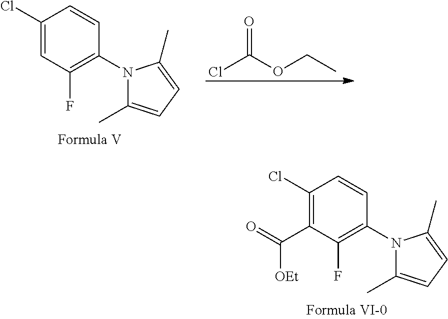

To a 3 L three-necked reaction flask equipped with a constant pressure dropping funnel was added the compound of Formula V (224 g, 1.00 mol) and dried tetrahydrofuran (1.3 L), was stirred for 5 minutes until the system was uniformly mixed. The reaction system was degassed by using nitrogen gas and cooled to −30° C., and thereto was added n-butyllithium solution (2.4 mol/L, 438 mL) slowly and dropwise upon keeping the reaction temperature of the system below −30° C. After the addition was completed, the system was stirred continuously for 1 hour at this temperature. Ethyl chloroformate (217 g, 2.00 mol) was dissolved in dried tetrahydrofuran (220 mL), degassed by using nitrogen gas, and cooled to −30° C. Then, to the above system was added the solution of ethyl chloroformate dropwise under the protection of nitrogen gas, during which the reaction temperature of the system was kept below −30° C. After the addition was completed, the system was stirred continuously for 30 minutes, and thereto was added a saturated aqueous solution of ammonium chloride (450 mL), and then the system was naturally warmed to room temperature. Ethyl acetate (1.5 L) and water (3.0 L) were added, and the phases were separated. The aqueous phase was extracted with ethyl acetate (1.5 L). The organic phase was combined, washed with saturated brine (2.0 L), and dried over anhydrous sodium sulfate. After the desiccant was filtered off, the solvent was removed through concentration in vacuo. The resulting residue was concentrated under reduced pressure to obtain a colorless and clear liquid (266 g, yield: 90%). The liquid product was rapidly solidified upon cooling.

¹H NMR (CDCl₃):δ7.33-7.23 (2H, m), 5.93 (2H, s), 4.47 (2H, q), 2.00 (6H, s), 1.43-1.39 (3H, m).

### Example 3

### Preparation of ethyl 3-amino-6-chloro-2-fluorobenzoate compound (Formula II-0)

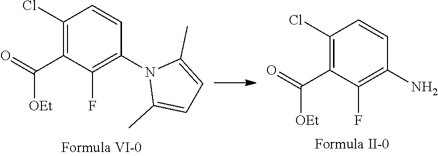 [SMILES: CCOC(=O)c1c(Cl)ccc(-n2c(C)ccc2C)c1F ; CCOC(=O)c1c(Cl)ccc(N)c1F]

To a 3 L three-necked reaction flask were added the compound of Formula VI-0 (286 g, 0.96 mol), ethanol (1.2 L) and water (400 mL), and uniformly stirred. Triethylamine (389 g, 3.84 mol) and hydroxylamine hydrochloride (997 g, 14.4 mol) were added. The reaction system was vigorously stirred at a temperature of 80° C. for 24 hours, concentrated under reduced pressure to remove most of ethanol. Water (3.0 L) and ethyl acetate (1.5 L) were added, and stirred, and the phases were separated. The resulting aqueous phase was extracted with ethyl acetate(1. L) twice. The organic phase was combined, washed with saturated brine (2 L), dried over anhydrous sodium sulfate, filtered and concentrated under reduced pressure to obtain a colorless liquid (140 g, yield: 90%).

¹H NMR (CDCl₃):δ 6.97 (1H, dd, J=8.8 Hz, J=0.8 Hz), 6.73 (1H, t, J=9.2 Hz), 4.44 (2H, q, J=6.8 Hz), 3.84 (2H, s), 1.41-1.38 (3H, m).

### Example 4

### Preparation of ethyl 6-chloro-2-fluoro-3-[3-fluoro-N-(3-fluoro-propylsulfonyl)propylsulfonamido]benzoate (Formula VIII-0)

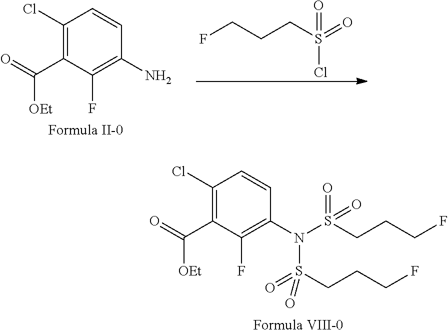 [SMILES: CCOC(=O)c1c(Cl)ccc(N)c1F ; O=S(=O)(Cl)CCCF ; CCOC(=O)c1c(Cl)ccc(N(S(=O)(=O)CCCF)S(=O)(=O)CCCF)c1F]

To a 3 L three-necked reaction flask were added the compound of Formula II-0 (162 g, 0.74 mol), triethylamine (244 g, 2.22 mol), and dichloromethane (970 mL), and stirred for 10 minutes until the system was uniformly mixed. Then 3-fluoro-1-propylsulfonyl chloride (244 g, 1.52 mol) was added slowly and dropwise, upon controlling the reaction temperature of the system below 20° C. After the addition was completed, the system was stirred at room temperature for 3 hours. The reaction solution was washed successively with 1 mol/L hydrochloric acid (2.5 L), water (2.0 L) and saturated brine (1.0 L), dried over anhydrous sodium sulfate, filtered and concentrated under reduced pressure to obtain a light brown liquid (329 g, 95%).

¹H NMR (CDCl₃):δ7.40-7.39 (1H, m), 7.35-7.28 (1H, m), 4.65-4.63 (2H, m), 4.53-4.44 (4H, m), 3.80-3.68 (4H, m), 2.37-2.27 (4H, m), 1.43-1.38 (3H, m).

### Example 5

### Preparation of 6-chloro-2-fluoro-3-(3-fluoro-propylsulfonamido)benzoic acid (Formula IX)

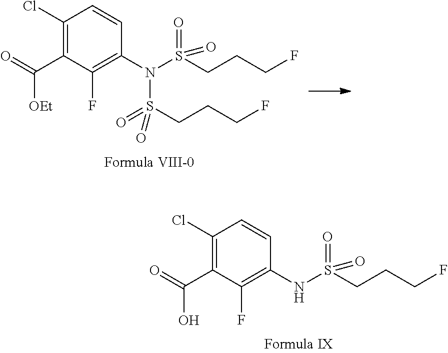 [SMILES: CCOC(=O)c1c(Cl)ccc(N(S(=O)(=O)CCCF)S(=O)(=O)CCCF)c1F ; O=C(O)c1c(Cl)ccc(NS(=O)(=O)CCCF)c1F]

To a 5 L three-necked flask were added the compound of Formula VIII-0 (309 g, 0.66 mol) and tetrahydrofuran (1.5 L), and stirred at room temperature for 10 minutes until the system was uniformly mixed. The system was cooled in an ice-water bath, and then 2 mol/L aqueous solution of potassium hydroxide (1.65 L, 3.3 mol) was added dropwise, during which the reaction temperature of the system was controlled below 10° C. The ice-water bath was removed after the addition was completed, and the system was stirred at room temperature for 3 days. The system was placed in an ice-water bath again, and then a concentrated hydrochloric acid was added dropwise until a pH value is less than 3, during which the temperature of the system was controlled below 10° C. The system was filtered, and the resulting solid was slurried in 2 L water, filtered again, washed with 2-3 L water, and dried to obtain the product (309 g, 95%).

¹H NMR (CDCl₃):δ10.04 (1H, s), 7.50 (1H, t, J=8.8 Hz), 7.39 (1H, dd, J=8.8 Hz, J=1.2 Hz), 4.60 (1H, t, J=5.6 Hz), 4.48 (1H, t, J=5.6 Hz), 3.28-3.24 (2H, m), 2.16-2.03 (2H, m).

### Example 6

### Preparation of ethyl 6-chloro-2-fluoro-3-(3-fluoro-propylsulfonamido)benzoate (Formula VIIIa-0)

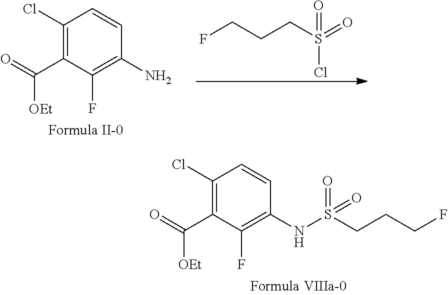 [SMILES: CCOC(=O)c1c(Cl)ccc(N)c1F ; O=S(=O)(Cl)CCCF ; CCOC(=O)c1c(Cl)ccc(NS(=O)(=O)CCCF)c1F]

To a 3 L three-necked reaction flask were added the compound of Formula II-0 (190 g, 0.87 mol), pyridine (345 g, 4.36 mol) and dichloromethane (1.9 L), and stirred for 10 minutes until the system was uniformly mixed. Then, a solution of 3-fluoro-1-propylsulfonyl chloride (168 g, 1.05 mol) in dichloromethane (0.34 L) was added slowly and dropwise, during which the reaction temperature of the system was controlled at 20-25° C. After the addition was completed, the system was warmed to 30° C., and reacted at this temperature for 24h. The reaction solution was washed successively with 4 mol/L hydrochloric acid (1.9 L) and saturated brine (1 L), and the organic phase was concentrated in vacuo to obtain a reddish brown liquid (298 g).

¹H NMR (CDCl₃):δ7.48 (1H, t), 7.36 (1H, dd), 4.64-4.62 (2H, m),4.58 (1H, t), 4.47 (1H, t), 3.28-3.24 (2H, m), 2.16-2.03 (2H, m), 1.41-1.38 (3H, m).

### Example 7

### Preparation of 6-chloro-2-fluoro-3-(3-fluoro-propylsulfonamido)benzoic acid (Formula IX)

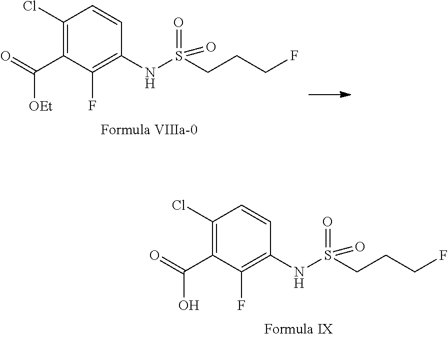 [SMILES: CCOC(=O)c1c(Cl)ccc(NS(=O)(=O)CCCF)c1F ; O=C(O)c1c(Cl)ccc(NS(=O)(=O)CCCF)c1F]

To a 5 L three-necked flask were added the compound of Formula VIIIa-0 (298 g, 0.87 mol) and tetrahydrofuran (1.5 L), and stirred at room temperature for 10 minutes until the system was uniformly mixed. The system was cooled to below 0° C., and 2 mol/L aqueous solution of potassium hydroxide (1.7 L) was added dropwise, during which the reaction temperature of the system was controlled below 10° C. After the addition was completed, the system was stirred at a temperature of 35° C. for 3 days, and then cooled, and subsequently the concentrated hydrochloric acid was added dropwise until a pH value is less than 3, during which the temperature of the system was controlled below 10° C. The phases were separated, and the aqueous phase was back-extracted with ethyl acetate (0.6 L) twice. The organic phase was combined, washed with saturated brine (0.9 L), concentrated in vacuo, and dried to obtain the product (259 g, 95%).

### Example 8

### Preparation of 6-chloro-2-fluoro-3-(3-fluoro-propylsulfonamido)benzamide (Formula X)

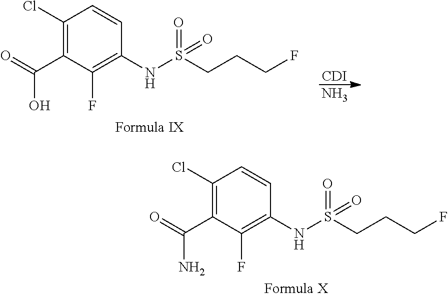

To a 3 L three-necked reaction flask were added the compound of Formula IX (239 g, 0.76 mol), N,N′-carbonyldiimidazole (184 g, 1.14 mol) and dried tetrahydrofuran (1.2 L), and heated under reflux for 1 hour. After the reaction was completed, the system was cooled to 0° C., and water (500 mL) was added. The system was stirred at 0° C., and ammonia gas was introduced into the system until the reaction was completed. 500 mL ice water was added, and the concentrated hydrochloric acid was added dropwise to adjust a pH value to 3-4, during which the temperature of the system was controlled to below 15° C. by using an ice-water bath. the phases were separated, and the aqueous phase was extracted with ethyl acetate (1 L) twice. The organic phase was combined, dried over anhydrous sodium sulfate, filtered, and concentrated under reduced pressure. The resulting residue was stirred for 2 hours after adding 240 mL ethanol, filtered, and dried to obtain the product (208 g, yield: 87%),of which the purity through HPLC detection is greater than 99% (area normalization method).

²H NMR (CDCl₃):δ9.98 (1H, s), 8.14 (1H, s), 7.87 (1H, s), 7.44-7.42 (1H, m), 7.35-7.32 (1H, m), 4.60 (1H, t, J=5.6 Hz), 4.48 (1H, t, J=5.6 Hz), 3.26-3.22 (2H, m), 2.14-2.06 (2H, m).

### Example 9

### Preparation of N-(3-amino-4-chloro-2-fluorophenyl)-3-fluoropropane-1-sulfonamide (Formula III)

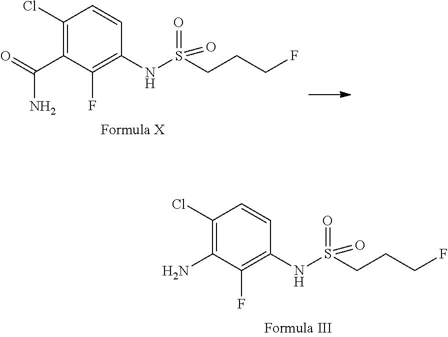 [SMILES: NC(=O)c1c(Cl)ccc(NS(=O)(=O)CCCF)c1F ; Nc1c(Cl)ccc(NS(=O)(=O)CCCF)c1F]

To a 3 L three-necked reaction flask were added the compound of Formula X (176 g, 0.56 mol) and tetrahydrofuran (880 ml), and stirred for 10 minutes until the system was uniformly mixed. The system was placed in an ice-water bath, and 4 mol/L aqueous solution of sodium hydroxide (1400 mL, 5.60 mol) was added, and then stirred for 10 minutes. An aqueous solution of sodium hypochlorite (1.05 L, 10% chlorine content) cooled below 5° C. was added portion-wise to the system over 5 minutes, and stirred for an additional 1 hour. The ice-water bath was removed, and the was naturally warmed to room temperature. The system was stirred for an additional 4-5 hours, and sodium thiosulfate (1.12 kg, 4.51 mol) was added, and stirred for 20 minutes until the sodium thiosulfate was completely dissolved. The reaction system was placed in an ice-water bath again, and then the concentrated hydrochloric acid was added dropwise to a pH value of 5-6, during which the temperature of the system was controlled below 20° C. The phases were separated, and the aqueous phase was extracted with ethyl acetate (700 mL) twice. The organic phase was combined, washed successively with water (1.0 L) and saturated brine (1.0 L), dried over anhydrous sodium sulfate, filtered and concentrated under reduced pressure. The resulting residue was slurried in 260 mL ethanol, and filtered. The filter cake was washed with about 50-60 ml ethanol, and dried to obtain a white solid (120 g, 75% yield), of which the purity through HPLC detection was greater than 99.5% (area normalization method).

¹H NMR (CDCl₃):δ9.66 (1H, s), 7.04 (1H, dd, J=8.4 Hz , J=1.6 Hz), 6.57 (1H, t, J=8.4 Hz), 4.60 (1H, t, J=6.0 Hz), 4.48 (1H, t, J=6.0 Hz), 3.19-3.16 (2H, m), 2.15-2.02 (2H, m).

1. A compound of Formula VI, or a salt or solvate thereof, 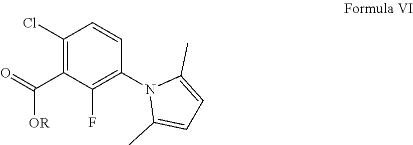
wherein R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted by one or more substituents selected from the group consisting of halogen, hydroxy, amino, lower alkyl, lower alkoxy and lower alkylthio.

2. A Compound of Formula VIII or Formula VIIIa, or a salt or solvate thereof, 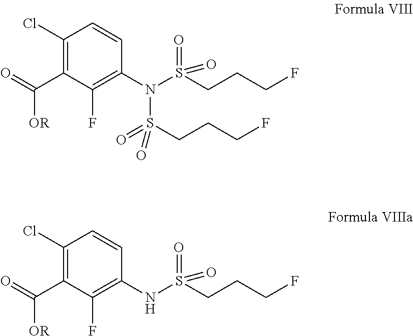
wherein R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and heteroaryl which is optionally substituted by one or more substituents selected from the group consisting of halogen, hydroxy, amino, lower alkyl, lower alkoxy and lower alkylthio.

3. A compound of Formula X, or a salt or solvate thereof,

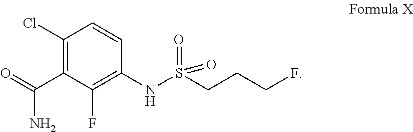 [SMILES: NC(=O)c1c(Cl)ccc(NS(=O)(=O)CCCF)c1F]

4. A method for preparing a compound of Formula III, comprising reacting a compound of Formula X according to claim 3 to prepare the compound of Formula III,

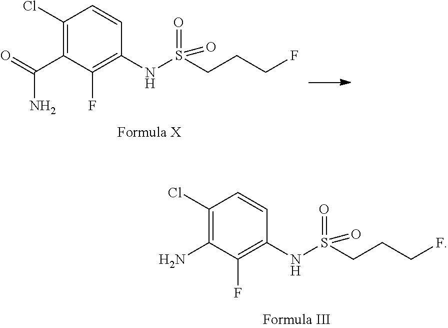 [SMILES: NC(=O)c1c(Cl)ccc(NS(=O)(=O)CCCF)c1F ; Nc1c(Cl)ccc(NS(=O)(=O)CCCF)c1F]

5. The method according to claim 4, further comprising reacting a compound of Formula IX with N,N′-carbonyldiimidazole (CDI), and then adding NH₃ to prepare the compound of Formula X,

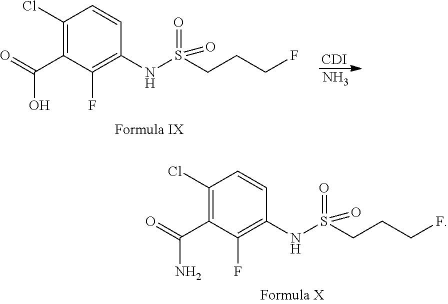

6. The method according to claim 5, further comprising reacting a compound of Formula VIII to prepare the compound of Formula IX, 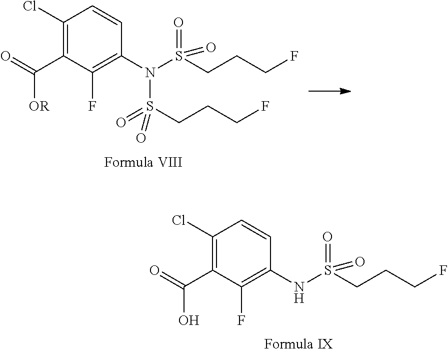
or
reacting a compound of Formula Villa to prepare the compound of Formula IX, 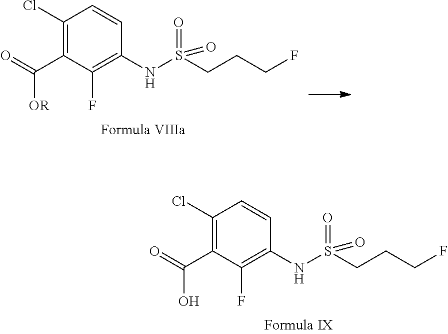
wherein each R is independently selected from the group consisting of alkyl, alkenyl, alkynyl, cycloalkyl, heterocycloalkyl, aryl, and heteroaryl, wherein each group is optionally substituted with one or more substituents selected from the group consisting of halogen, —OH, —NH₂, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and aryl and heteroaryl both of which are optionally substituted by one or more substituents selected from the group consisting of halogen, hydroxy, amino, lower alkyl, lower alkoxy and lower alkylthio.

7. The method according to claim 6, further comprising reacting a compound of Formula II with 3-fluoro-1-propylsulfonyl chloride to prepare the compound of Formula VIIIa, 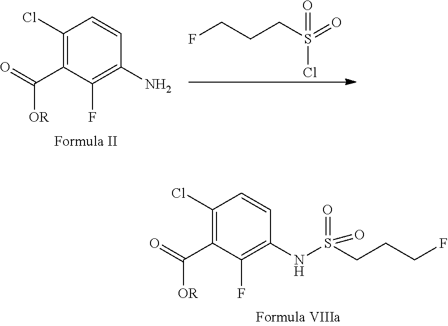
wherein R is as defined in claim 6,
or
reacting the compound of Formula II with 3-fluoro-1-propylsulfonyl chloride to prepare the compound of Formula VIII, 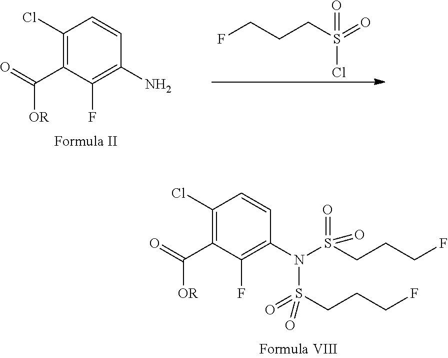
wherein R is as defined in claim 6.

8. The method according to claim 7, wherein further comprising reacting a compound of Formula VI to prepare the compound of Formula II: 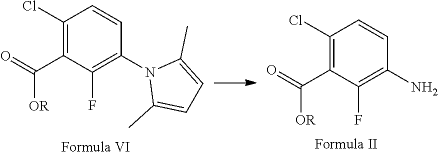
wherein R is as defined in claim 6.

9. The method according to claim 8, wherein further comprising:
(1) reacting a compound of Formula IV with 2,5-hexanedione to prepare a compound of Formula V, and
(2) reacting the compound of Formula V with a compound of Formula VII to prepare the compound of Formula VI, 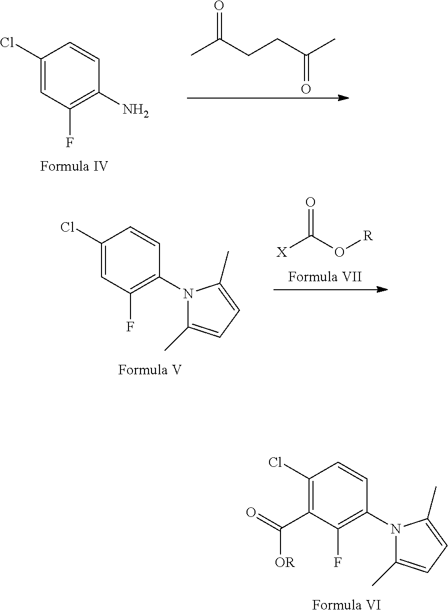
wherein R is as defined in claim 6, and X is selected from halogen.

10. The method according to claim 7, further comprising the following steps:
(1) reacting a compound of Formula IV with 2,5-hexanedione to prepare a compound of Formula V,
(2) reacting the compound of Formula V with a compound of Formula VII to prepare a compound of Formula VI, and
(3) reacting the compound of Formula VI to prepare the compound of Formula II, 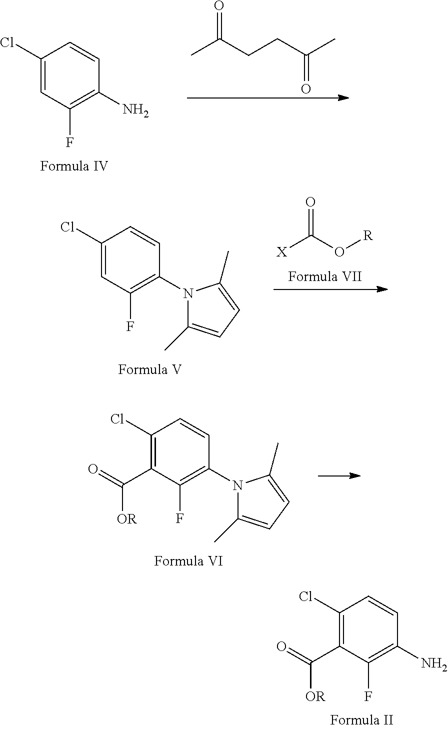
wherein R is as defined in claim 6, and X is selected from halogen.

11. The method according to claim 10, wherein in step (1), the compound of Formula IV is reacted with 2,5-hexanedione in the presence of a catalyst.

12. The method according to claim 10, wherein in step (3), the compound of Formula VI is reacted in the presence of a reagent selected from the group consisting of hydroxylamine hydrochloride, hydroxylamine hydrochloride and a base, and hydrochloric acid and a base, wherein hydrochloric acid is first added to react for a period of time, and then the base was added, when the reagent is hydrochloric acid and the base.

13. A method for preparing a compound of Formula VIII according to claim 2, comprising reacting a compound of Formula II with 3-fluoro-1-propylsulfonyl chloride to prepare the compound of Formula VIII, 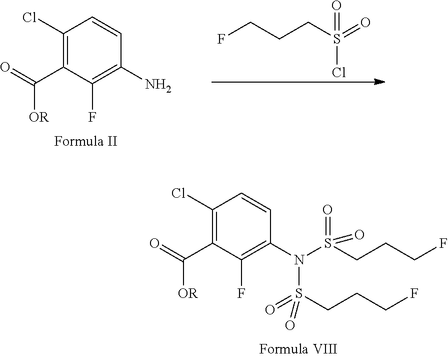
wherein R is as defined in claim 2.

14. A method for preparing a compound of Formula VIIIa according to claim 2, comprising reacting a compound of Formula II with 3-fluoro-1-propylsulfonyl chloride to prepare the compound of Formula VIIIa, 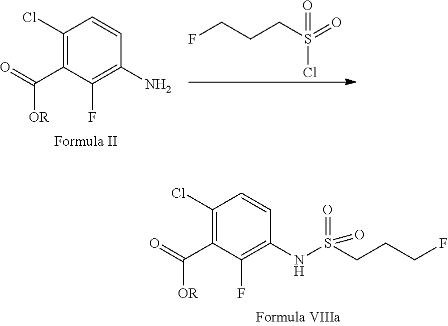
wherein R is as defined in claim 2.

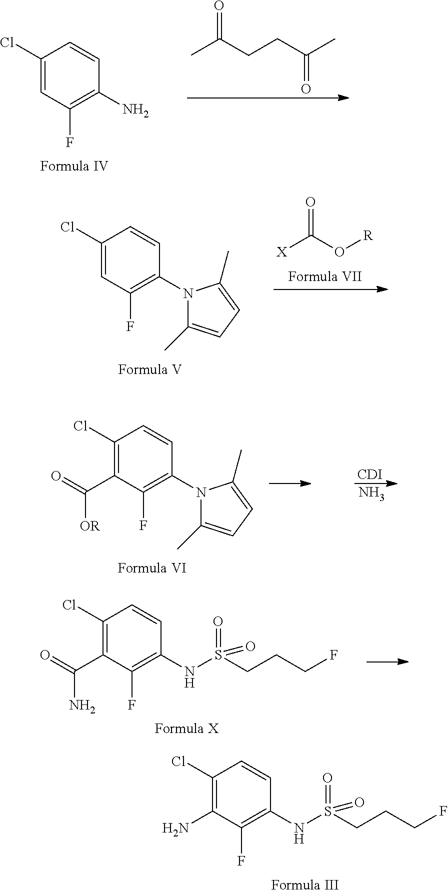

15. The method according to claim 5, further comprising reacting a compound of Formula VIII to prepare the compound of Formula IX, 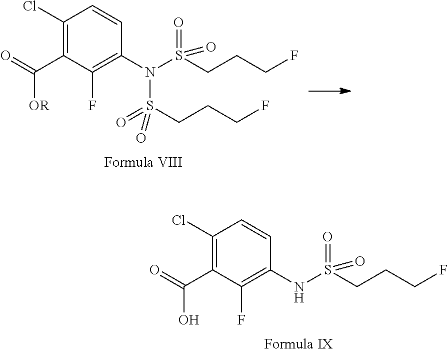
or
reacting a compound of Formula VIIIa to prepare the compound of Formula IX, 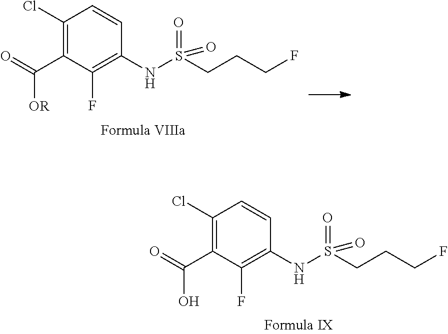
wherein each R is selected from the group consisting of alkyl, alkenyl, alkynyl, cycloalkyl, heterocycloalkyl, aryl, and heteroaryl, wherein each group is optionally substituted with one or more substituents selected from the group consisting of halogen, lower alkyl, halogen-substituted lower alkyl, cycloalkyl, halogen-substituted cycloalkyl, lower alkoxy, halogen-substituted lower alkoxy, lower alkylthio, halogen-substituted lower alkylthio, mono-alkylamino, di-alkylamino, cycloalkylamino, and heteroaryl which is optionally substituted by one or more substituents selected from the group consisting of halogen, hydroxy, amino, lower alkyl, lower alkoxy and lower alkylthio.

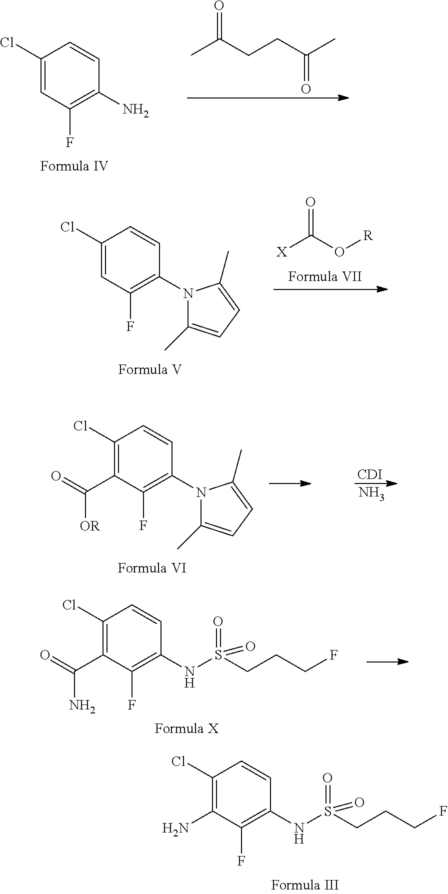

16. A method for preparing a compound of Formula X according to claim 3, comprising reacting a compound of Formula IX with N,N′-carbonyldiimidazole, and then adding NH₃ to prepare the compound of Formula X,

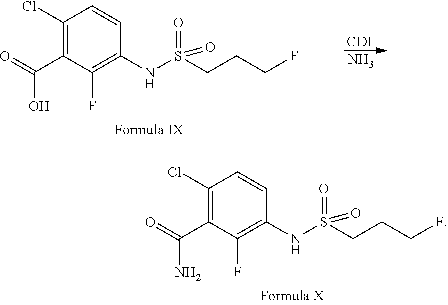

17. The method according to claim 4, further comprising reacting a compound of Formula IX with N,N′-carbonyldiimidazole, and then adding NH₃ to prepare a compound of Formula X

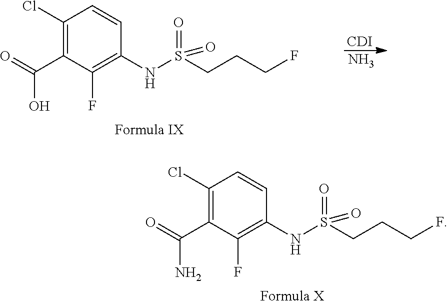

18. The method according to claim 4, further comprising the following steps:
(1) reacting a compound of Formula IV with 2,5-hexanedione to prepare a compound of Formula V,
(2) reacting the compound of Formula V with a compound of Formula VII to prepare a compound of Formula VI,
(3) reacting the compound of Formula VI to prepare a compound of Formula II,
(4) reacting the compound of Formula II with 3-fluoro-1-propylsulfonyl chloride to prepare a compound of Formula VIII,
(5) reacting the compound of Formula VIII to prepare a compound of Formula IX, and
(6) reacting the compound of Formula IX with N,N′-carbonyldiimidazole, and then adding NH₃ to prepare a compound of Formula X,

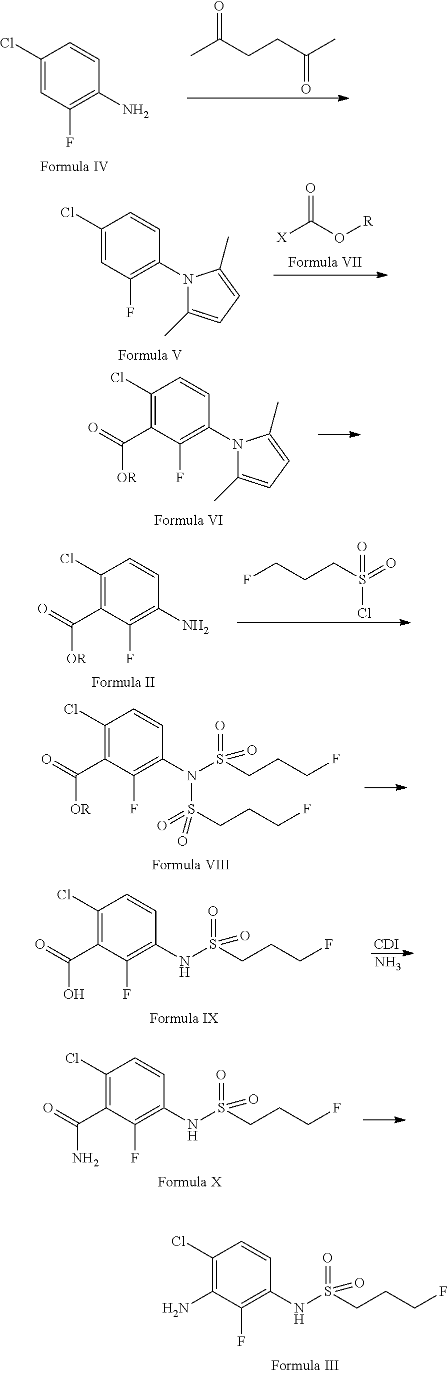

19. The method according to claim 4, further comprising the following steps:
(1) reacting a compound of Formula IV with 2,5-hexanedione to prepare a compound of Formula V,
(2) reacting the compound of Formula V with a compound of Formula VII to prepare a compound of Formula VI,
(3) reacting the compound of Formula VI to prepare a compound of Formula II;
(4) reacting the compound of Formula II with 3-fluoro-1-propylsulfonyl chloride to prepare a compound of Formula VIIIa,
(5) reacting the compound of Formula VIIIa to prepare a compound of Formula IX, and
(6) reacting the compound of Formula IX with N,N′-carbonyldiimidazole, and then adding NH₃ to prepare a compound of Formula X, [chemical structure]
